(19) United States
(12) Patent Application Publication
(10) Pub. No.: US 2013/0052134 A1
(43) Pub. Date: Feb. 28, 2013

(54) PEG-PLGA-PLL POLYMER AND METHOD FOR PREPARING AND USING THE SAME AS THE DRUG AND GENE CARRIER

(71) Applicants: Yourong Duan, Shanghai (CN); Peifeng Liu, Shanghai (CN); Ying Sun, Shanghai (CN); Ming Shen, Shanghai (CN); Mingjie Zhu, Shanghai (CN); Yanming Sun, Shanghai (CN); Qi Wang, Shanghai (CN); Zelai He, Shanghai (CN); Liubin Qin, Shanghai (CN); Hui Yu, Shanghai (CN); Xiaoyu Li, Shanghai (CN); Xuelian Qi, Shanghai (CN); Xiaoyan Chen, Shanghai (CN); Qiufen Sun, Shanghai (CN); Bingwu Wang, Shanghai (CN)

(72) Inventors: Yourong Duan, Shanghai (CN); Peifeng Liu, Shanghai (CN); Ying Sun, Shanghai (CN); Ming Shen, Shanghai (CN); Mingjie Zhu, Shanghai (CN); Yanming Sun, Shanghai (CN); Qi Wang, Shanghai (CN); Zelai He, Shanghai (CN); Liubin Qin, Shanghai (CN); Hui Yu, Shanghai (CN); Xiaoyu Li, Shanghai (CN); Xuelian Qi, Shanghai (CN); Xiaoyan Chen, Shanghai (CN); Qiufen Sun, Shanghai (CN); Bingwu Wang, Shanghai (CN)

(73) Assignee: Shanghai Cancer Institute, Shanghai (CN)

(21) Appl. No.: 13/518,874

(22) PCT Filed: Dec. 28, 2010
(86) PCT No.: PCT/CN2010/002180

§ 371 Date: Jun. 22, 2012

(30) Foreign Application Priority Data
Dec. 30, 2009 (CN) 200910247576.7

(51) Int. Cl. A61K 47/34; A61K 49/00; C08G 63/91; A61K 9/14; B82Y 5/00

(52) U.S. Cl. 424/9.1; 525/448; 424/400; 514/772.1; 525/54.1; 525/54.2; 977/773; 977/906

(57) ABSTRACT

This invention belongs to the nanotechnology field, and discloses a nano drug delivery system with polyethyleneglycol-poly(lactic-co-glycolic acid)-poly-L-lysine (PEG-PLGA-PLL) polymer as the skeleton. The carrier can have the function of passive targeting through control of the carrier particle size. The polymer skeleton is modified through introducing side chains and specific targeting groups, so as to adjust and improve the carrier performance, and enable the carrier to have the function of active targeting. Such carrier material also has the functions of transporting active substances, tumor treatment and diagnosis, ultrasonic contrast, reversing or reducing drug resistance and so on. It is mainly applied to (1) Targeting preparation of anticancer drugs; (2) preparation to reverse or reduce the drug resistance of the tumor; (3) reagent for tumor diagnosis and contrast; (4) reagent to transfect DNA plasmids; (5) pharmaceutical preparation for cancer gene therapy; (6) reagent used to transfect antisense nucleic acid and siRNA (RNA interference); (7) pharmaceutical preparation used to prepare antisense nucleic acid and siRNA (RNA interference).

### TECHNICAL FIELD

The present invention relates to the technical field of tumor targeting delivery and carrier for the sustained-release drug delivery system, and particularly, to the drug and gene delivery system with PEG-PLGA-PLL cationic polymer as the carrier, its preparation and application in medicines.

### BACKGROUND ART

Clinical application of chemotherapeutic drugs in the malignant tumor treatment has achieved certain success in many cases, but at the same time, still has some serious problems. The major problem is that the chemotherapeutic drugs are generally less selective, which results in the occurrence of serious dose-dependent poisonous side effect, and greatly restricts the clinical curative effect of chemotherapeutic drugs. Another problem is the fast occurrence of drug resistance among tumor cells. Therefore, developing the therapeutic method of specifically targeting tumor cells and minimizing the damage to normal cells is of very important significance and broad application prospect.

In the recent decades, the targeting delivery carrier can effectively enhance the curative effect due to its unique advantages, and attracts much domestic and foreign attention as a consequence. Especially, since the rapid development of the delivery system with the biodegradable polymer as the carrier, the targeting delivery carrier can effectively reduce the poisonous side effect of drugs, delay the in vivo drug metabolism, and improve the curative effect. Many biodegradable materials such as the poly(lactic acid), poly(lactic-co-glycolic acid) and so on have been widely used as the delivery carrier for drugs, genes and imaging agents, which has made certain progress.

Most nano carriers are recognized and ingested by the macrophages before arriving at the target sites in vivo, so the curative effect cannot be achieved; as a result, many domestic and foreign scholars attach mPEG to the carrier surface, so that long chain of the mPEG can allow the nano carrier to effectively escape from the ingestion of the reticuloendothelial system, thereby achieving the purpose of long cycle, and obtaining better curative effect. The degradable polymer poly(lactic-co-glycolic acid) (PLGA) may be slowly degraded in vivo, allowing the drug to be slowly released with the material degradation, thereby achieving the curative effect for a long time. This material has been approved by the U.S. FDA. The cationic polymer poly-L-lysine (PLL) has good biodegradability, and its degradation product is the amino acid required for the human body. The PLL compound still bears the positive charge with flexible and stable structure, its molecular weight is easy to be adjusted, and its polymer skeleton may be modified through introducing side chains and specific targeting groups, so as to further adjust and improve the carrier performance, and achieve the purpose of sustained release of drugs. The combination of PLGA and PLL can exert the advantages of both. Therefore, the nano drug delivery system with the mPEG-PLGA-PLL cationic polymer as the skeleton is a very excellent carrier for sustained-release drugs.

Targeting ability of the drug delivery system is the key to accurately delivering the active substances to the target sites, and the nano drug delivery system with the mPEG-PLGA-PLL cationic polymer as the skeleton can better solve this problem. Diameter of the developed nano carrier system may be maintained at 1 nm-10μ. There is no gap between the blood vessels around normal tissues, but there is a gap of about 100 nanometer between the blood vessels around tumor tissues, therefore the nanoparticles will penetrate from these gaps, and gather at the tumor site using the enhanced permeability and retention effect, then attack the cancer cell, but will not damage the normal cells, thereby achieving the effect of passive targeting. After the nanoparticles are modified using the targeting groups, the targeting groups may specifically combine with the target sites, the active targeting effect formed by the receptor-mediated targeting drug delivery system allows the anticancer drugs to be quite accurately delivered to the tumor cells, so as to realize the targeting treatment of malignant tumors. The nano drug delivery system with the mPEG-PLGA-PLL cationic polymer designed in the present invention as the skeleton can simultaneously connect the targeting groups and load more than two active substances, thereby achieving the purpose of targeting delivery and multiple therapeutic methods.

### SUMMARY OF THE INVENTION

The present invention is intended to provide a nano drug delivery carrier system with the mPEG-PLGA-PLL cationic polymer as the skeleton. Wherein, PEG has the long cycle effect, PLGA is biodegradable and has the sustained-release effect, and PLL bearing the positive charge can mediate the combination with genes bearing the negative charge. The carrier can have the function of passive targeting through controlling its particle size. The polymer skeleton is modified through introducing side chains and specific targeting groups, so as to adjust and improve the carrier performance, and allow the carrier to have the function of passive targeting. This carrier material also has the functions of transporting active substances, tumor treatment and diagnosis, ultrasonic contrast, reversing or reducing drug resistance and so on.

Technical matters to be solved in this invention are to synthesize appropriate carriers, so that different targeting groups can be effectively grafted on the carriers, and load active substances, so as to achieve effective targeting delivery of the active substances to the targeting sites.

The cationic polymer mPEG-PLGA-PLL defined in the invention is a series of materials with different molecular weights and different monomer proportions, molecular weight of the mPEG-PLGA-PLL cationic polymer is 1.0×10³-9.0×10⁶; molar ratio of the said PEG to PEG-PLGA-PLL polymer is 1-30:100-1; molar ratio of the said PLL to the PEG-PLGA-PLL is 1-40:100-1; molar ratio the of said PLGA to PEG-PLGA-PLL is 1-30:100-1; and molar ratio of the said PEG to PLGA to PLL is 1-30:1-30:1-40.

The polymer material mPEG-PLGA defined in this invention is synthesized through ring-opening polymerization. The catalyst used in the synthesis includes stannous octanoate, zinc lactate, stannous chloride or p-toluenesulfonic acid.

The synthetic method of the mPEG-PLGA-PLL defined in this invention comprises 5 steps as follows:

(1) Preparation of the mPEG-PLGA:

In a closed tube under vacuum, mPEG, lactide and glycolide are catalyzed at 100˜250° C. for 2˜100 hrs. Wherein, molar ratio of the said lactide to glycolide is 1˜100:100˜1, mass percent of the mPEG in the gross mass of the raw material is 1%˜50%, molecular weight of the said mPEG is 350˜20000, mass percent of the catalyst in the gross mass of the raw material is 0.0001˜1%, and the said catalyst is stannous octanoate, zinc lactate, stannous chloride or p-toluenesulfonic acid;

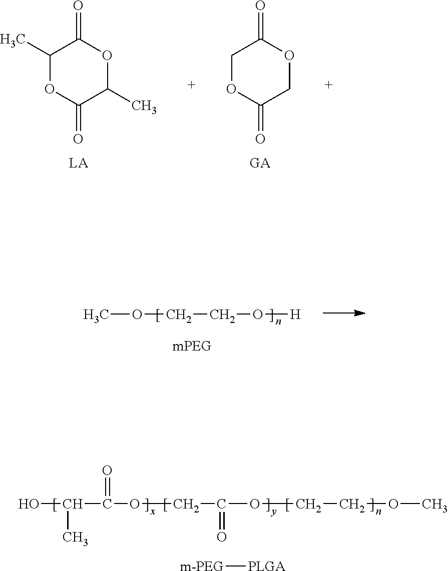

(2) Preparation of the mPEG-PLGA-[(N-tert-butoxycarbonyl)-L-phenylalanine]:

The product in the step (1), tert-butoxycarbonyl-L-phenylalanine, N,N-dicyclohexylcarbodiimide and 4-dimethylpyridine are reacted in an organic solvent under nitrogen protection at room temperature for 0.5˜5 days; molar ratio of the said product in the step (1), N-tert-butoxycarbonyl-L-phenylalanine, N,N-dicyclohexylcarbodiimide and 4-dimethylpyridine is 1:0.01˜30:0.01˜30:0.01˜30;

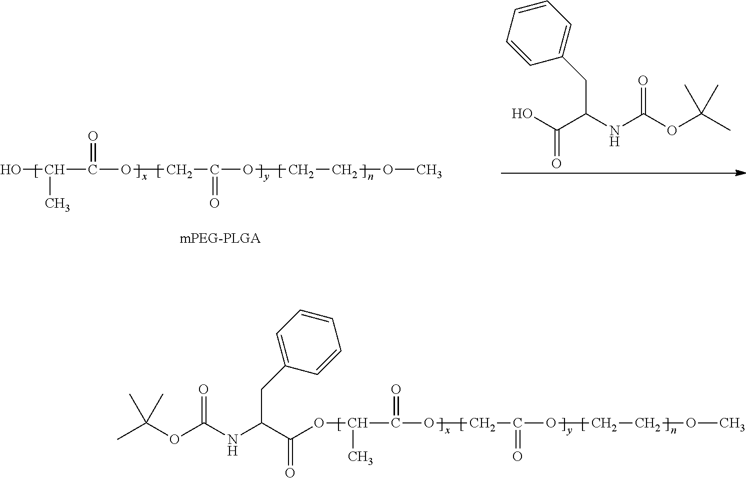 [SMILES: COCCOC(=O)COC(=O)C(C)O ; CC(C)(C)OC(=O)NC(Cc1ccccc1)C(=O)O ; COCCOC(=O)COC(=O)C(C)OC(=O)C(Cc1ccccc1)NC(=O)OC(C)(C)C]

(3) Preparation of the mPEG-PLGA-L-phenylalanine

The product in the step (2) is reacted with trifluoroacetic acid in an organic solvent under nitrogen protection at −20° C.˜40° C. for 0.1˜24 hours; molar ratio of the said product in the step (2) to the trifluoroacetic acid is 1:0.01˜30;

(4) Preparation of the mPEG-PLGA-[(N-benzyloxycarboxylic)-PLL]

The product in the step (3) is reacted with N-carboxyanhydrides of amino acids under nitrogen protection in an organic solvent at room temperature for 1˜6 days; molar ratio of the product in the step (3) to the N-carboxyanhydrides of amino acids is 1:0.01˜100;

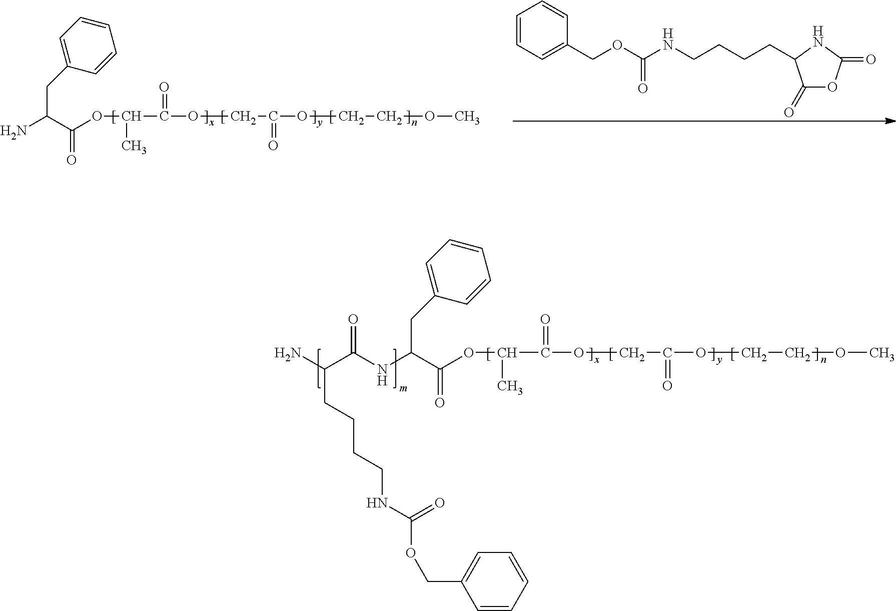

(5) Preparation of the mPEG-PLGA-PLL

The product in the step (4) is reacted with a certain amount of 33% hydrobromic acid-glacial acetic acid solution at 0° C. for 0.1˜24 hours; molar ratio of the said product in the step (4) to 33% hydrobromic acid-glacial acetic acid solution is 1:0.01˜100;

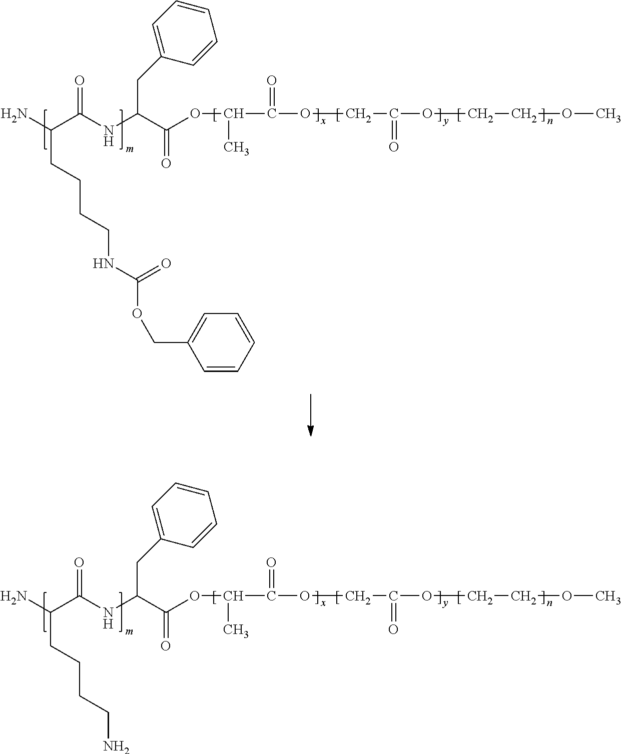

The polymer mPEG-PLGA-PLL defined in this invention is an excellent drug carrier used to load drugs and prepare sustained release nanoparticles using a mechanical mixer, an ultrasonic machine and a high-pressure homogenizer. The resulting nanoparticles are controllable below 1 nm-10 μm (preferably 10 nm-1000 nm), are characterized by smooth surface, good uniformity, regular particles without conglutination, good redispersibility, high drug-loading rate and high encapsulation rate, and are used to prepare sustained release nanoparticles for intravenous injection or intramuscular injection or oral administration. The resulting nanoparticles can be dispersed in solid, semi-solid or solution, and are preferably made into pharmaceutical preparations for injection, especially for intravenous injection.

The said targeting group that can be connected with the polymer mPEG-PLGA-PLL in this invention includes the peptide inhibiting the tumor angiogenesis; anticancer angiogenesis factors such as the fibroblast growth factors (FGFs) and vascular endothelial growth factors (VEGF); active groups such as the polypeptide, folic acid, antibody, transferrin, carbohydrate, and polysorbate; targeting groups with suitable modifiable functional groups and the derivatives thereof, such as the glycyrrhizic acid, glycyrrhetinic acid, cholic acid, low density lipoprotein (LDL), hormone, nucleic acid and so on. The RGD peptide is any straight-chain or annular polypeptide fragment containing arginine-glycine-aspartic acid sequence, including the tripeptide, tetrapeptide, pentapeptide, hexapeptide, heptapeptide, octapeptide, nonapeptide and decapeptide containing the RGD sequence, or the straight-chain or annular polypeptide fragment containing RGD mimetic (RGDm); the adopted antibody encapsulates many antibodies including EGFR; the carbohydrates include galactose, chitosan, mannan, amylopectin and glucan and so on; the grafting rate of the targeting groups is 0.0001%-50%.

The loaded active substance defined in the invention includes the drug, gene, contrast agent for diagnosis, gas inside the microbubbles and probe. The drug includes any anticancer drug that is applicable to be made into a nanoparticle drug delivery system, such as organic anticancer drugs, water-soluble anticancer drugs or water-insoluble anticancer drugs, for instance, antifolate drugs (such as methotrexate), purine drugs (such as mercaptopurine), antipyridine drugs (such as fluorouracil and tegafur), ribonucleotide reductase inhibitor (such as hydroxyurea), DNA polymerase inhibitor (such as cyclocytidine), drugs directly affecting and damaging the DNA structure and function (such as nitrogen mustard, cyclophosphamide, formylmerphalan, cisplatin, mitomycin C and camptothecin), protein synthesis inhibitors (such as adriamycin, L-asparaginase, daunomycin and mithramycin), drugs affecting the tubulin assembly and spindle fiber formation (such as the vincristine and etoposide), and active contrast agent or diagnostic agent used for imagers such as the nuclear magnetic resonance imager, ultrasonic imager, CT and so on, and the diagnostic agent is respectively used for ultrasonic imaging, nuclear magnetic resonance imaging, CT and PET. The gene includes the therapeutic genes, such as SiRNA, suicide gene, antioncogene, antisense nucleic acid and so on; gas inside the microbubbles includes the microbubble ultrasound contrast agents such as the air, fluorocarbon gas, sulfur hexafluoride and so on.

The said polymer carrier in this invention loaded with the gas inside the microbubble can be used for ultrasonic contrast, solid tumor localization, and auxiliary tumor treatment through the ultrasonic cavitation effect.

The said nano drug delivery system with the PEG-PLGA-PLL polymer as the skeleton in this invention can simultaneously connect at least one or more than one different targeting groups or/and load at least one or more than one different active substances, thereby achieving the purpose of targeting delivery, drug and gene therapy and combining multiple therapeutic methods.

The said drug-loaded nanoparticle system with the PEG-PLGA-PLL polymer as the skeleton in this invention may be prepared with the following methods:

Multiple emulsion method: The synthesized polymer modified with polypeptide is dissolved in ethyl acetate, dichloromethane or the organic solvent mixture of dichloromethane and acetone, to which aqueous solution of the drugs is added, and then the primary emulsion is obtained through ultrasonic or high-pressure homogenization. Afterwards, water dispersion medium is added and emulsified to obtain the secondary emulsion, the organic phase of which is removed through agitation or rotary evaporation to obtain the nanoparticle suspension.

Co-precipitation method: The synthesized polymer modified with polypeptide is dissolved in acetone together with the drugs, the resulting solution is added to the water dispersion medium dropwise while stifling, and the nanoparticle suspension is obtained through completely evaporating the organic solvent by agitation.

Emulsified solvent diffusion process: The synthesized polymer modified with polypeptide is dissolved in the solvent mixture of acetone and dichloromethane together with the drugs, the resulting solution is added to the water dispersion medium, then the emulsion is obtained through ultrasonic or high-pressure homogenization and emulsification, and finally the nanoparticle suspension is obtained through completely evaporating the organic solvent at room temperature.

The preparation method of the nanometer drug delivery system with the PEG-PLGA-PLL polymer as the carrier defined in this invention, wherein, the said dispersion medium is the surfactant applicable to preparing nanoparticles, such as the dextran 40, dextran 70, Pluronic F68 or polyvinyl alcohol (PVA) and so on, and the dispersion medium is at the concentration of 0.1˜20% (w/v).

The said organic solvent used to prepare nanoparticles in this invention include the organic solvent that is applicable to preparing nanoparticles, such as ethyl acetate, dichloromethane, acetone, alcohol and so on.

The nanometer drug delivery system with the PEG-PLGA-PLL polymer as the carrier defined in this invention, wherein, the application in respect of the drug carrier includes the drug delivery, long cycle, biodegradation, sustained and controlled release, passive targeting, active targeting, transporting active substances, tumor treatment and diagnosis, ultrasonic contrast, reversal of the drug resistance among tumor cells, and disease diagnosis and treatment. The drug delivery approach of the said drug delivery system in preparing the drugs to treat corresponding diseases includes injection, oral administration and mucosal administration.

The nanometer drug delivery system with the PEG-PLGA-PLL polymer as the carrier defined in this invention, wherein, the said system may be made into the lyophilized preparation for preservation and application, the lyophilized supporting agent includes the fucose, glucose, lactose, sucrose, dextran, sorbitol, mannitol and PEG and so on. The supporting agent is at the concentration of 0.01-20% (w/v).

The preparation method for the nanometer drug delivery system in this invention is simple, and suitable for large scale production, especially for preparation of targeted nanoparticle drug delivery system for anticancer drugs.

The drug-loading nanoparticles prepared for the nano drug delivery system with the mPEG-PLGA-PLL polymer as the skeleton defined in this invention has the function of reversing or reducing the drug resistance of tumors.

### BRIEF DESCRIPTION OF THE DRAWINGS

FIG. 1 shows the NMR spectrum of mPEG-PLGA-PLL (DMSO is dimethylsulfoxide) as described in Example 1.

FIG. 2 is a nanoparticle size distribution diagram with the particle size as the abscissa, and intensity as the ordinate as described in Example 2: 2(a) is a diagram showing the nanoparticle size distribution diagram of mPEG-PLGA-PLL-RGD nanoparticles; 2(b) is a diagram showing the nanoparticle size distribution of mitoxantrone-loaded mPEG-PLGA-PLL-RGD nanoparticles (Nicomp™-380ZLS particle size analyzer).

FIG. 3 shows the observation of the appearance of siRNA-loaded mPEG-PLGA-PLL nanoparticles with an atomic force microscope as described in Example 5, where the white points are the appearance of the nanoparticles.

FIG. 4 shows the cytotoxicity experimental results of mPEG-PLGA-PLL as described in Example 9.

FIG. 5 shows the drug ingestion experiment of the drug-resistant cell MCF-7/MIT promoted by drug-loaded mPEG-PLGA-PL nanoparticles as described in Example 10.

FIG. 6 shows the ultrasound/microbubble-promoted RPE-J cell ingestion of Cy3-siRNA-loaded nanoparticles as described in Example 14.

FIG. 7 is a diagram showing the drug distribution in animal tissues in vivo. The distribution of the drug-loaded nanoparticles in animal tissues in vivo is measured at 4th hour, 28th hour, and 51st hours after tail intravenous injection of the mitoxantrone-loaded mPEG-PLGA-PLL-RGD nanoparticles as described in Example 16.

FIG. 8 is a diagram of the animal tumor growth inhibited by drug-loaded nanoparticles. The normal saline group, free mitoxantrone, mitoxantrone-loaded mPEG-PLGA-PLL nanoparticles and mitoxantrone-loaded mPEG-PLGA-PLL-RGD nanoparticles are administered through tail intravenous injection once every four days, then the tumor volume is measured, and the treatment duration is 36 days. FIG. 8(a) shows the growth curve of the tumor volume, and FIG. 8(b) shows the tumor photos after treatment as described in Example 16.

### DESCRIPTION OF THE SYMBOLS

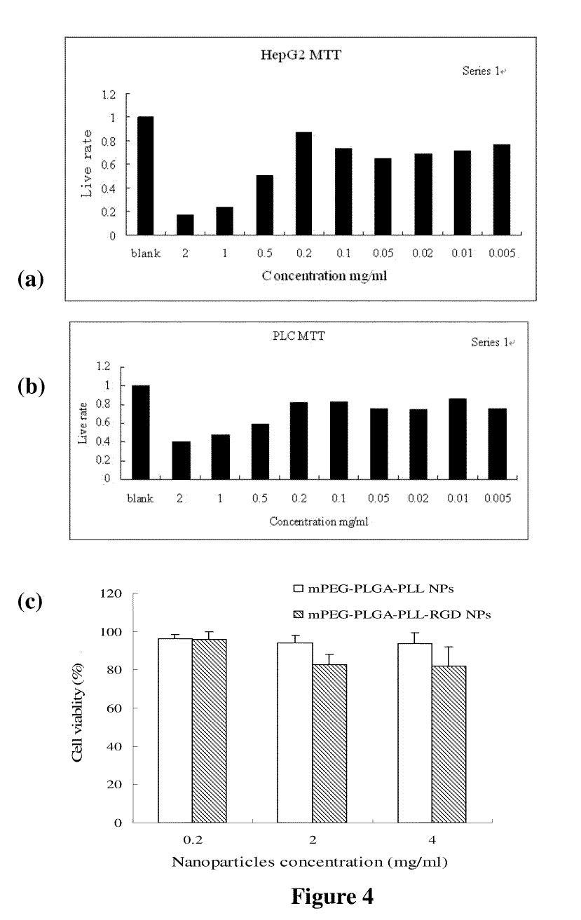

In FIG. 4, Nanoparticles concentration represents the concentration of the nanoparticles; Live rate represents the survival rate of cells; cell viability represents the survival rate of cells expressed as %. HepG2 represents the cell viability of the liver cancer cell HepG2 measured with MTT method, and PLC MTT represents the cell viability of the liver cancer cell PLC measured with MTT method. MDA-MB-231 represents the mammary cancer cells.

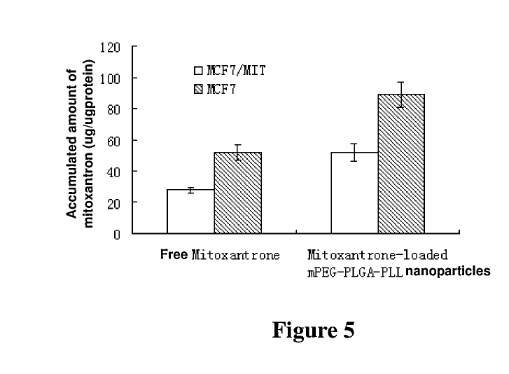

In FIG. 5, opposite to the mitoxantrone-loaded mPEG-PLGA-PLL nanoparticles, free mitoxantrone represents the mitoxantrone without a carrier. MCF7 represents mammary cancer cells; MCF7/MIT represents the MCF7 cells resistant to mitoxantrone.

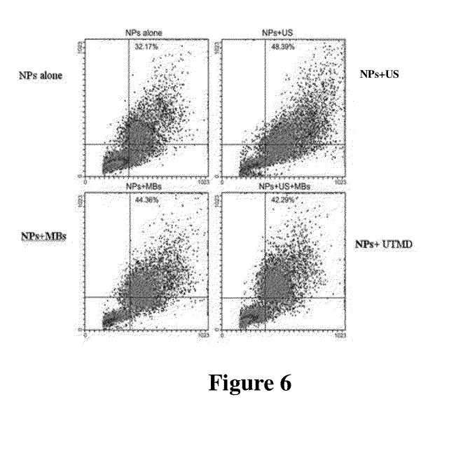

In FIG. 6, NPs alone is the sole nanoparticle group; NPs+US represents the nanoparticle+ultrasound group; NPs+MBs represents the nanoparticle+microbubble group; NPs+UTMD represents the nanoparticle+microbubble and ultrasound group.

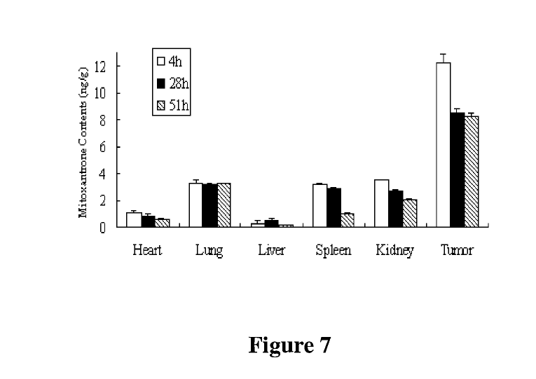

In FIG. 7, Mitoxantone Contents represent the content of mitoxantrone. 4 h represents the amount of mitoxantrone in the animal tissue at 4th hour; 28 h represents the amount of mitoxantrone in the animal tissue at 28th hour; 51 h represents the amount of mitoxantrone in the animal tissue at 51th hour.

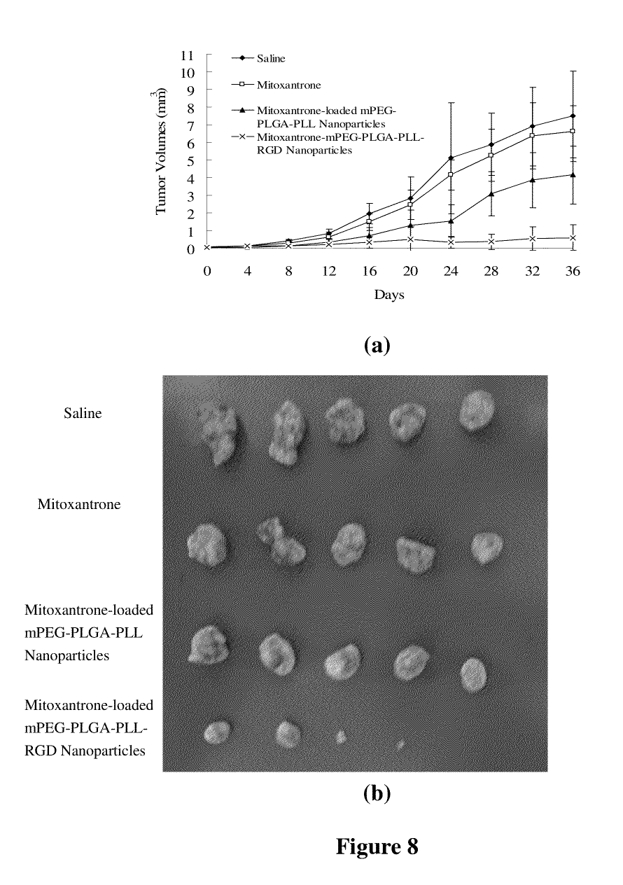

In FIG. 8, saline represents normal saline; mitoxantrone represents the anticancer drug of mitoxantrone; (a) the number of days is the abscissa, and tumor volume is the ordinate.

### DETAILED DESCRIPTION OF THE INVENTION

This invention illustrates the carrier used for drug/gene delivery, as well as its preparation method and application. This invention is not limited to the specific configuration, methods, steps and substances disclosed in this document, because such configuration, methods and substances may be changed. The terminologies used in the invention are just provided to illustrate the detailed description of embodiments of the invention, and are not intended to limit the invention, as the scope of this invention will be only limited by the appended claims and equivalent contents thereof.

In the description and appended claims, both “a” and “the said” in a single form includes corresponding contents in the plural form, unless otherwise clearly described in the context.

The following examples are provided to further illustrate this invention, and are not intended to limit the content of this invention.

### Example 1

### Synthesis of mPEG-PLGA-PLL

(1) Preparation of mPEG-PLGA-PLL: 17.28 g of lactide and 3.48 g of glycolide (molar ratio was 8:2), as well as 10% mass percent of mPEG (relative to the gross mass of the raw materials) with the molecular weight of 2K, were added to a heat-resistant glass tube that was vacuumed and dried through heating, and then zinc lactate catalyst was added. The resulting mixture was insufflated with nitrogen; dissolved through heating and vacuumed; cooled, solidified, and vacuumed for 2 hours; then sealed at 150° C. for 40 hours.

30.24 g of lactide and 10.44 g of glycolide (molar ratio was 8:2), as well as 20% mass percent of mPEG (relative to the gross mass of the raw materials) with the molecular weight of 5K, were added to a heat-resistant glass tube that was vacuumed and dried through heating, and then zinc lactate catalyst was added. The resulting mixture was insufflated with nitrogen; dissolved through heating and vacuumed; cooled, solidified, and vacuumed for 2 hours; then sealed at 120° C. for 5 days.

(2) Preparation of mPEG-PLGA-tert-butoxycarbonyl-L-phenylalanine: 6 g of mPEG-PLGA was dissolved in a dry organic solvent, and then 1.06 g of tert-butoxycarbonyl-L-phenylalanine, 0.83 g of N,N-dicyclohexylcarbodiimide and 0.08 g of 4-dimethylpyridine were added while stirring. The resulting mixture was stirred under nitrogen protection at room temperature for 2 days, filtered, and then washed with sodium bicarbonate solution 3 times and with water 3 times. The organic phase was collected, dried with anhydrous magnesium sulphate, and concentrated. Then the desired product was precipitated with glacial ethyl ether, filtered and dried under vacuum.

(3) Preparation of mPEG-PLGA-tert-butoxyamino-L-phenylalanine: 2.6 g of product in the above (2) was dissolved in a dry organic solvent, and then 5.2 ml of dry trifluoroacetic acid was added dropwise under nitrogen protection at 0° C. for 30 min. Another 2 hours later, the solvent and unreacted trifluoroacetic acid was removed through rotary evaporation. The residue was dissolved in an organic solvent, and then washed with sodium bicarbonate solution 3 times and with water 3 times. The organic phase was collected, dried with anhydrous magnesium sulphate, concentrated, precipitated with glacial ethyl ether, filtered and dried under vacuum.

(4) Preparation of mPEG-PLGA-protected L-phenylalanine: 2 g of product in the above (3) was dissolved in a dry organic solvent, and then 1.6 g of N-carboxyanhydrides (NCA) of amino acids was added. The mixture was kept under nitrogen protection at room temperature for 3 days, concentrated, precipitated with glacial ethyl ether, filtered and dried under vacuum.

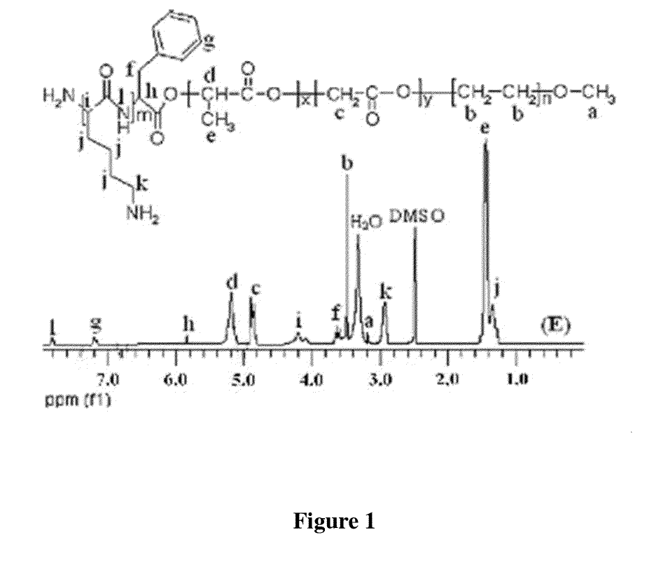

(5) Preparation of mPEG-PLGA-PLL: 1 g of product in the above (4) was dissolved in 3 ml of trifluoroacetic acid, and then 5 ml of 33% (v/v) hydrobromic acid (HBr) in acetic acid was added. The mixture was kept at 0° C. for 1 hour, precipitated with glacial ethyl ether, filtered and dried under vacuum. Please see FIG. 1 for the NMR spectrum.

(6) Grafting of cRGD: 400 mg of mPEG-PLGA-PLL was dissolved in DMSO, and then 46 mg of cRGD and 27 mg of N,N′-carbonyl diimidazole (CDI) were added. The mixture was stirred under nitrogen protection at room temperature for 4 hours. On completion of the reaction, the solution was placed in a dialysis bag for 24 hours, and then preserved by lyophilization.

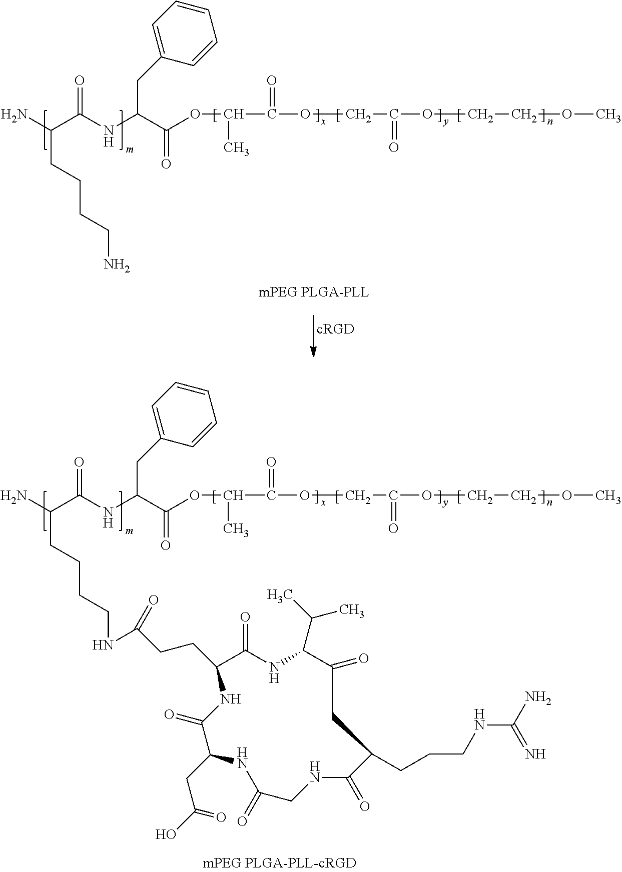

Grafting of folic acid: 28 mg of folic acid was dissolved in DMSO, 0.1 g of copolymer mPEG-PLGA-PLL was added, and then 30 mg of 1-(3-dimethylaminopropyl)-3-ethylcarbodiimide hydrochloride (EDC) was added. The mixture was stirred for 4 hours, then dialysed with deionized water, lyophilized and sealed for application.

Grafting of the antibody: 5 mg of antibody was dissolved in DMSO, 15 mg of EDC and 10 mg of N-hydroxysuccinimide (NHS) were added while stifling, and then 0.1 g of copolymer mPEG-PLGA-PLL was added. The mixture was stirred for 4 hours, then dialysed with deionized water, lyophilized and sealed for application.

Grafting of transferrin: 2 mg of transferrin was dissolved in DMSO, 10 mg of EDC and 10 mg of N-hydroxysuccinimide (NHS) were added while stifling, and then 0.1 g of copolymer mPEG-PLGA-PLL was added. The mixture was stirred for 4 hours, then dialysed with deionized water, lyophilized and sealed for application.

### Example 2

### Preparation of Mitoxantrone-Loaded mPEG-PLGA-PLL Nanoparticles

Preparation with the emulsification evaporation method: 8 mg of mPEG-PLGA-PLL was dissolved in 400 μL of dichloromethane, and then 40 μL of 10 mg/mL aqueous solution of mitoxantrone chloride was added to the resulting solution. After ultrasonic emulsification, 4.4 mL of 1 wt % aqueous solution of poloxamer F68 was added, followed by ultrasonic emulsification for the 2nd time and stifling at room temperature for 3 hours. Then the nanoparticle suspension was obtained by removing the organic phase. The resulting nanoparticle size was controlled at 10-1000 nm.

Preparation with the membrane emulsification method: 8 mg of mPEG-PLGA-PLL and 0.4 mg of mitoxantrone chloride were dissolved in 400 μL of acetone, and then the membrane was formed through rotary evaporation. Afterwards, 4 mL of aqueous solution was added, and stirred at room temperature for 3 hours, then the nanoparticle suspension was obtained. The resulting nanoparticle size was controlled at 10-1000 nm.

Preparation with the dialysis method: 8 mg of mPEG-PLGA-PLL was dissolved in 3 mL of DMSO, and then 0.4 mg of mitoxantrone chloride was added and stirred until uniform; afterwards, the organic solution was added to water while stirring, then the solution was packed into the dialysis bag for 48 hours, thus the nanoparticle suspension was obtained after removing the organic solvent. The resulting nanoparticle size was controlled at 10-1000 nm.

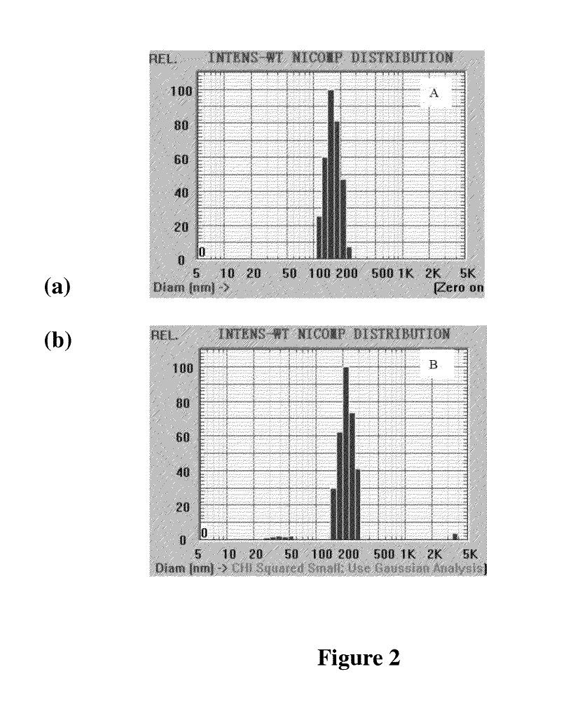

Preparation with the interfacial precipitation method: 8 mg of mPEG-PLGA-PLL and 0.4 mg of mitoxantrone chloride were dissolved in 400 μL of acetone, and then 4 mL of 2 wt % PVA solution was added to the above solution while stirring at a certain speed. Then the nanoparticle suspension was obtained after removing the acetone through pressurized evaporation. The resulting nanoparticle size was controlled at 10-1000 nm. (FIG. 2)

### Example 3

### Preparation of DNA-Loaded mPEG-PLGA-PLL Nanoparticles

Preparation with the emulsification evaporation method: 8 mg of mPEG-PLGA-PLL was dissolved in 400 μL of dichloromethane, and the resulting solution was added to 4.4 mL of 1 wt % aqueous solution of F68 for ultrasonic emulsification, followed by stifling at room temperature for 3 hours. Then the nanoparticle suspension was obtained by removing the organic phase. An appropriate amount of mPEG-PLGA-PLL nanoparticle solution was added to equivalent volume of plasmid DNA solution while fully stifling, and the resulting mixture was incubated at low temperature for 30 min to obtain the DNA gene-loaded nanoparticles.

Double emulsion solvent evaporation method, also known as the solvent evaporation method, means that the gene dissolved in water was taken as the internal water phase, and 8 mg of mPEG-PLGA-PLL dissolved in 400 μL of dichloromethane was taken as the oil phase, both form the primary water-in-oil (W/O) emulsion after supersonic emulsification, then 4 mL of 2 wt % aqueous solution of polyvinyl alcohol was poured into the primary emulsion, and emulsified again to the secondary water-in-oil-in-water (W/O/W) emulsion. Microballs were solidified during the organic solvent evaporation through agitation, followed by centrifugal washing, vacuum drying, and radiation sterilization at 60° C.

### Example 4

### Preparation of the Gene/Drug-Loaded mPEG-PLGA-PLL Nanoparticles

Preparation with the emulsification evaporation method: 8 mg of mPEG-PLGA-PLL was dissolved in 400 μL of dichloromethane, and then 40 μL of 10 mg/mL aqueous solution of mitoxantrone chloride was added to the resulting solution. After ultrasonic emulsification, 4.4 mL of 1 wt % aqueous solution of F68 was added, followed by ultrasonic emulsification for the 2nd time and stifling at room temperature for 3 hours. Then the nanoparticle suspension was obtained by removing the organic phase. An appropriate amount of mPEG-PLGA-PLL nanoparticle solution was added to equivalent volume of plasmid DNA solution while fully stirring, and the resulting mixture was incubated at low temperature for 30 min to obtain the gene-loaded and drug-loaded nanoparticles.

### Example 5

### Preparation of mPEG-PLGA-PLL Nanoparticles Loaded with the siRNA (Small Interference RNA)

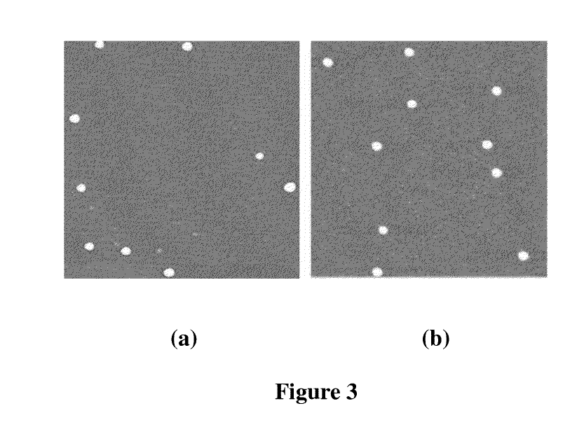

8 mg of mPEG-PLGA-PLL was dissolved in 400 μL of dichloromethane to form the organic phase, which was mixed with the water phase, i.e. 50 μL of 20 μmol/L siRNA-Cy3 (Cy3-labelled siRNA) solution. Then the primary emulsion was obtained through ultrasonic emulsification, and poured into 4.4 mL of external water phase containing 0.5 wt % F68 emulsifier. Secondary emulsion was obtained through further ultrasonic emulsification, and then the nanoparticle dispersion system mPEG-PLGA-PLL-siRNA-Cy3 was obtained through completely evaporating the organic solvent via rotary evaporation. (FIG. 3)

### Example 6

### Preparation of mPEG-PLGA-PLL Nanoparticles Loaded with the Gas Inside the Microbubbles

Preparation with the double emulsion method: 12 mg of mPEG-PLGA-PLL was dissolved in 1 mL of dichloromethane while fully stifling until completely dissolved (as the continuous phase), and then 200 μL of double distilled water (as the dispersion phase) was added. Milk white emulsion (W/O microballs) was obtained after ultrasonic emulsification, then the emulsion (dispersion phase) was poured into 4 mL 2 wt % PVA solution (continuous phase), and homogenized with a homogenizer (W/O/W microballs). Afterwards, the resulting mixture was added to 4 mL of isopropanol solution, and stirred at high temperature, allowing the microballs to be solidified on the surface, and dichloromethane to be naturally volatilized as far as possible. After washing with double distilled water (DDW) and n-hexane many times, the microballs were centrifugated (to remove the dichloromethane), collected, dried at room temperature, uniformly mixed with an appropriate amount of DDW, and placed in a vacuum freeze drier at −45° C. for 48 hours. Then air suction was stopped, and octafluoropropane was slowly insufflated into the freeze drying chamber until reaching the atmospheric pressure. The ultrasonic contrast agent of mPEG-PLGA-PLL microbubbles was obtained after keeping the gas valve of the freeze drier closed for 8 hours.

### Example 7

### Preparation of the Mitoxantrone-Loaded mPEG-PLGA-PLL Nanoparticles Modified with Folic Acid

9 mg of mPEG-PLGA-PLL modified with folic acid was dissolved in 400 μL of dichloromethane, and then 40 μL of 10 mg/mL aqueous solution of mitoxantrone chloride was added to the resulting solution. After ultrasonic emulsification, 4.4 mL of 1 wt % aqueous solution of F68 was added, followed by ultrasonic emulsification for the 2nd time and stirring at room temperature for 3 hours. Then the nanoparticle suspension was obtained by removing the organic phase. The resulting nanoparticle size was controlled at 10-1000 nm.

### Example 8

### Preparation of the Mitoxantrone-Loaded mPEG-PLGA-PLL Nanoparticles Modified with EGFR

8 mg of mPEG-PLGA-PLL was dissolved in 400 μL of dichloromethane, and then 40 μL of 10 mg/mL aqueous solution of mitoxantrone chloride was added to the resulting solution. After ultrasonic emulsification, 4.4 mL of 1 wt % aqueous solution of F68 was added, followed by ultrasonic emulsification for the 2nd time and stirring at room temperature for 3 hours. Then the nanoparticle suspension was obtained by removing the organic phase; afterwards, 10 mg of EDC and 5 mg of NHS were added to the nanoparticle solution, and stirred for 2 hours, then EGFR antibody was added to the solution, and stirred for 4 hours to obtain the nanoparticles modified with EGFR antibody.

### Example 9

### Research on the Cytotoxicity of mPEG-PLGA-PLL

HepG2 and PLC liver cancer cells were spread on a 96-well microplate with the cell density in each well of 5×10⁴/ml, and incubated in a cell incubator with 5% volume fraction of carbon dioxide at 37° C. overnight. The mPEG-PLGA-PLL nanoparticles with the quantity of nanoparticles in the range of 0.05-200 μg were added to the 96-well microplate (The cell group only with the addition of culture solution was taken as the negative control group), and 4 duplicate wells were established for each experimental condition. 20 μL of 5 mg/mL 3-(4,5-dimethylthiazolyl-2)-2,5-diphenyltetrazolium bromide (trade name MTT) was added at 24th h after incubation, and the incubation was kept for another 4 hours, afterwards the culture solution was dissolved in 100 μL of DMSO. The reading at 490 nm of the microplate reader was recorded to calculate the cell viability according to the formula, and 4 duplicate wells were established for each experimental condition.

The MTT experiment proved that HepG2 and PLC liver cancer cells had high viability (up to above 85%) when the mPEG-PLGA-PLL concentration was 0.2 mg/ml, and the mPEG-PLGA-PLL material had equivalent cytotoxicity to both liver cancer cells. The cytotoxicity of the material was low (FIG. 4)

The effect on the growth of mammary cancer cells MDA-MB-231 was observed with similar method. (FIG. 4)

### Example 10

### Drug Ingestion Experiment of the Drug-Resistant Cell MCF-7/MIT Promoted by Nano Drug Delivery System

Mammary cancer cells MCF-7 and the cell strains resistant to mitoxantrone chloride (MIT) (MCF-7/MIT) were offered as a gift by the State Key Laboratories of Oncogenes and Related Genes of Shanghai Cancer Institute. The MCF-7 cell and MCF-7/MIT cells were spread on a 24-well microplate with 80000 cells/well, and incubated in a cell incubator with 0.5 ml of culture solution containing 5% carbon dioxide at 37° C. overnight. After cell attachment, the culture solution was respectively changed to 0.5 ml of culture solution containing free mitoxantrone chloride or mitoxantrone chloride-loaded nanoparticles (concentration of mitoxantrone chloride is 20 ug/ml), the incubation was kept for another 1 hour, the culture solution was discarded, and the residue was washed with phosphoric acid buffer solution twice, then the cells were lysed with cell lysis solution. The collected lysate was divided into two shares, one share was used to detect the ultraviolet absorbance at 610 nm and calculate the concentration of mitoxantrone chloride, and another share was used to detect the protein content with 2-carboxyquinoline or bicinchoninic acid (BCA) protein assay kit. Finally, the intracellular drug concentration was calculated and marked with the protein content. 3 duplicate wells were established for each condition.

The experimental result indicated that 1 hour after respective administration of free mitoxantrone chloride and mitoxantrone chloride-loaded nanoparticles with the same drug concentration, drug ingestion in the MCF-7 cell was respectively 29.28±0.45 ug/ug protein and 55.21±0.95 ug/ug protein, (P<0.05), and the latter was about 1.9 times as much as the former; drug ingestion in the MCF-7/MIT cell was respectively 37.48±2.50 ug/ug protein and 87.30±2.97 ug/ug protein, (P<0.05), and the latter was about 2.4 times as much as the former. (FIG. 5)

### Example 11

### Experiment of the Ingestion of Cy3-siRNA-Loaded mPEG-PLGA-PLL Nanoparticles Among Liver Cancer Cells

Experimental method: HepG2 and PLC liver cancer cells at the logarithmic growth phase were spread on glass bottom dishes with 2×10⁴ HepG2 and PLC liver cancer cells on each dish, and incubated in a cell incubator with 0.3 ml of culture solution containing 5% carbon dioxide at 37° C. overnight. After cell attachment, 600 of Cy3-siRNA-loaded mPEG-PLGA-PLL nanoparticles were added to each well, allowing the final concentration of mPEG-PLGA-PLL nanoparticles to be 0.2 mg/ml. The resulting system was mixed uniformly, and incubated in a cell incubator with 5% carbon dioxide at 37° C. for 4 hours. The operating fluid was extracted, and washed with phosphoric acid buffer solution at 4° C. three times, then the nucleus was stained with 4′,6-diamidino-2-phenylindole (DAPI) for 5˜10 min, followed by washing with phosphoric acid buffer solution 3 times. 100 μl of phosphoric acid buffer solution was added to each well for detection using confocal laser scanning microscopy.

The experimental result indicated that both HepG2 and PLC liver cancer cells could better ingest mPEG-PLGA-PLL nanoparticles after co-incubation with the nanoparticles for 4 hours, and ingestion among the HepG2 group was more significantly increased than that among the PLC group.

### Example 12

### Experiment of the Ingestion of Cy3-siRNA-Loaded Nanoparticles Among PC-3, RPE-J and QGY-7701 Cells

The experimental method was the same that in Example 11. The experiment was intended to observe the ingestion of Cy3-siRNA-loaded nanoparticles among PC-3, RPE-J and QGY-7701 cells. The research findings indicated that all above cells can ingest nanoparticles.

Note: PC-3 was prostate cancer cells, RPE-J was the retinal pigment epithelial cells in rats, and QGY-7701 was human liver cancer cells.

### Example 13

### Experiment of the Ingestion of Cy3-siRNA-Loaded Nanoparticles Among RPE-J Cells at Different Time

The experimental method was the same that in Example 11. The experiment was intended to observe the ingestion of Cy3-siRNA-loaded nanoparticles among RPE-J cells at different time. The research findings indicated that internalization speed of nanoparticles among RPE-J cells of the high concentration group was significantly higher than that of the low concentration group.

### Example 14

### Ultrasound/Microbubble-Promoted RPE-J Cell Ingestion of Cy3-siRNA-Loaded Nanoparticles

RPE-J cells were planted on every other line and every other well of a 24-well microplate with 1×10⁵ cells/well, and incubated in a thermostatic incubator with 5% carbon dioxide overnight, allowing cell attachment. Every two holes were classified into one group with the same experimental parameters, and digested cells in two wells were collected and processed as one sample. Each experiment was repeated three to five times.

The probe of the ultrasonic therapy apparatus Topteam 161 provided by the US Chattanooga Corporation had the sectional area of 25 mm², frequency of 1 MHz, and pulse repetition frequency of 100 Hz. The fixed ultrasonic probe was coated with the coupling agent with the thickness of 2˜3 mm on its surface, and the microplate was positioned on the probe, composing the ultrasonic circuit of probe-coupling agent-cell plate-cells.

The commercialized SonoVue® of Italian Bracco Corporation was composed of the sulfur hexafluoride gas surrounded by the phospholipid shell. According to the user manual, 5 ml of normal saline was extracted with an aseptic injector to dilute SonoVue® powder, and the resulting mixture was fully and uniformly mixed by manually shaking. Microbubbles had the mean diameter of 2.5 μm and concentration of (2˜5×10⁸) microbubbles/ml.

According to the experimental design, nanoparticles were first added to the RPE-J cell wells, statically incubated in a thermostatic incubator containing 5% CO₂ for 10 min, and then ultrasonic irradiation was conducted immediately after the addition of microbubbles. On completion of the ultrasonic irradiation, cells were incubated in a thermostatic incubator with 5% CO₂ for 24 hours. The culture medium was discarded, and the residue was eluted with phosphoric acid buffer solution (PBS) three times. 300 μl of fresh culture medium was added to maintain the cell growth environment, and the fluorescence uptake in RPE-J cells was observed with an inversed fluorescence microscope (Axiovert S 100, German ZEISS Corporation). The culture medium was discarded, 0.3˜0.5 ml of 0.25% trypsin was added, followed by digestion and cell collection. 450 g of the resulting mixture was centrifugated (Centrifuge 5810R, German Eppendorf Corporation) for 5 minutes. The uptake rate and fluorescence intensity in RPE-J cells were detected with a flow cytometer (Facs Calibur, the US Becton Dickeinson Corporation).

24 hours later, the flow cytometer proved that ultrasound/microbubbles may effectively deliver the Cy3-labelled gene-loaded mPEG-PLGA-PLL nanoparticles to RPE cells. (FIG. 6)

### Example 15

### Distribution of Cy5-siRNA-Loaded mPEG-PLGA-PLL Nanoparticles in Animal Tissues In Vivo

Each nude mouse was given with 0.1 nmol of Cy5-siRNA-loaded mPEG-PLGA-PLL nanoparticles through intravenous injection, and the distribution in tissues in vivo was observed at different time.

The result indicated that mPEG-PLGA-PLL nanoparticles were mainly distributed in the liver, lung and tumor of the nude mouse in vivo; mPEG-PLGA-PLL nanoparticles had better passive targeting effect for the liver and lung; mPEG-PLGA-PLL nanoparticles were concentrated at the tumor site, which assumed a good concentration effect within 72 hours; mPEG-PLGA-PLL nanoparticles had not only the targeting effect of tissues and organs, but also the sustained-release function.

The experimental result showed that the tissues can be targeted through controlling the nanoparticle size.

### Example 16

### Research on the Distribution of Mitoxantrone-Loaded Nanoparticles in Mice Bearing MDA-MB-231 Mammary Cancer In Vivo and its Treatment

0.2 ml of MDA-MB-231 mammary cancer cells (2×10⁵) was subcutaneously injected to the axillary region near the forelimb of nude mice, and 2 weeks later, the tumor model was successfully established. Afterwards, the mice bearing human mammary cancer cells (MDA-MB-231) were randomly divided into 3 groups with 4 mice in each group, then mitoxantrone-loaded mPEG-PLGA-PLL-RGD nanoparticles were injected, and these animals were killed respectively at 2nd hour, 4th hour and 51st hour. Their heart, liver, spleen, lung, kidney and tumor tissues were taken out, weighed and ground to extract the drugs inside them. The chromatographic column was tested according to the following HPLC conditions: Kromasil 100-5C₁₈ (250 mm×4.6 mm ID), guard column: AUTO Science C.270A, column temperature: 30° C., mobile phase: methanol: 0.16 mol. L⁻¹ ammonium acetate buffer solution (PH2.7) (48:52), flowing rate: 1.0 ml·min⁻¹, detection wave length: 599 nm, sampling volume: 10 μl.

As can be seen from the experimental result, the concentration was low in some animal tissues such as the heart, liver, and lung, showing that nanoparticles had good biocompatibility, and can effectively escape from the ingestion of reticuloendothelial system. But the drug concentration in tumor tissues was far higher than that in other organs, and slightly decreased as the time goes by, but was still maintained at a higher level, indicating that mitoxantrone-loaded mPEG-PLGA-PLL-RGD nanoparticles can effectively target the tumor tissues. (FIG. 7)

In the therapeutic test, corresponding normal saline, free mitoxantrone, drug-loaded mPEG-PLGA-PLL nanoparticles and drug-loaded mPEG-PLGA-PLL-RGD nanoparticles were given once every 4 days through tail intravenous injection, the tumor volume was subsequently measured, and the treatment duration was 36 days. The experimental result showed that with the extension of the treatment time, the tumor volume in the group of drug-loaded mPEG-PLGA-PLL-RGD nanoparticles was obviously lower than that in the groups of free mitoxantrone and drug-loaded mPEG-PLGA-PLL nanoparticles; the group of drug-loaded mPEG-PLGA-PLL-RGD nanoparticles had a significantly higher tumor inhibition rate (91.27%) than the free mitoxantrone group (10.32%) and the group of drug-loaded mPEG-PLGA-PLL nanoparticles (42.06%), indicating that the drug-loaded mPEG-PLGA-PLL-RGD nanoparticles had a significant tumor inhibition effect on the mice bearing MDA-MB-231 mammary cancers, as shown in FIG. 8.

1-14. (canceled)

15. A polyethyleneglycol-poly(lactic-co-glycolic acid)-poly-L-lysine polymer, wherein the polymer is a polyethyleneglycol-poly(lactic-co-glycolic acid)-poly-L-lysine cation polymer having a molecular weight of about 1.0×10³ to 9.0×10⁶ Dalton; a molar ratio of polyethyleneglycol (PEG) in the polymer to the polymer is about 1:( 1/30) to 1:100, a molar ratio of poly-L-lysine (PLL) to the polymer is about 1:( 1/40) to 1:100, a molar ratio of poly(lactic-co-glycolic acid) (PLGA) to the polymer is about 1:( 1/30) to 1:100, and a molar ratio of the PEG to PLGA to PLL in the polymer is about (1-30):(1-30):(1-40).

16. The polyethyleneglycol-poly(lactic-co-glycolic acid)-poly-L-lysine polymer as defined in claim 15, wherein the PEG has a methylated hydroxyl group at one end and has the molecular weight of about 0.5K-20K Dalton, the PLGA has the molecular weight of about 1K-100K Dalton, and the PLL has the molecular weight of about 0.5K-100K.

17. The polyethyleneglycol-poly(lactic-co-glycolic acid)-poly-L-lysine polymer as defined in claim 16, wherein the PEG has the molecular weight of about 1K-6K Dalton, the PLGA has the molecular weight of about 1K-50K Dalton, and the PLL has the molecular weight of about 1K-20K Dalton.

18. A method for synthesizing the polyethyleneglycol-poly(lactic-co-glycolic acid)-poly-L-lysine polymer as defined in claim 15, comprising
synthesizing an mPEG-PLGA through a ring-opening polymerization,
reacting a phenylalanine having tert-butoxycarbonyl-protected amino groups with hydroxyl group at one end of the mPEG-PLGA to obtain an mPEG-PLGA-[(N-tert-butoxycarbonyl)-L-phenylalanine],
removing protection of the amino groups on the mPEG-PLGA-[(N-tert-butoxycarbonyl)-L-phenylalanine] to obtain mPEG-PLGA-L-phenylalanine,
polymerizing the mPEG-PLGA-L-phenylalanine in a closed reaction system with N-carboxyanhydrides of amino acids prepared via triphosgene to obtain a PEG-PLGA-PLL cationic polymer,
deaminating the PEG-PLGA-PLL cationic polymer to obtain the PEG-PLGA-PLL polymer.

19. The method for preparing the polyethyleneglycol-poly(lactic-co-glycolic acid)-poly-L-lysine polymer as defined in claim 18, wherein the mPEG-PLGA is prepared by reacting mPEG, lactide, and glycolide as starting material in presence of a catalyst in a closed reaction system under vacuum at about 100° C. to 250° C. for about 2 hours to 100 hour,
wherein a molar ratio of the lactide to the glycolide is about 1:0.01 to 1:100, a mass percent of the mPEG in a total mass of the starting material is about 1% to 50%, a molecular weight of the mPEG is about 350 to 20000 Dalton, a mass percent of the catalyst in a total mass of the starting material is about 0.0001% to 1%, and the catalyst is stannous octanoate, zinc lactate, stannous chloride, or p-toluenesulfonic acid.

20. The method for preparing the polyethyleneglycol-poly(lactic-co-glycolic acid)-poly-L-lysine polymer as defined in claim 18, wherein the mPEG-PLGA-[(N-tert-butoxycarbonyl)-L-phenylalanine is prepared by
reacting the mPEG-PLGA, a tert-butoxycarbonyl-L-phenylalanine, N,N-dicyclohexylcarbodiimide, and 4-dimethylpyridine in an organic solvent under nitrogen protection at room temperature for about 0.5 to 5 days,
wherein a molar ratio of the mPEG-PLGA, the N-tert-butoxycarbonyl-L-phenylalanine, the N,N-dicyclohexylcarbodiimide, and the 4-dimethylpyridine is 1:(0.01˜30):(0.01˜30):(0.01˜30).

21. The method for preparing the polyethyleneglycol-poly(lactic-co-glycolic acid)-poly-L-lysine polymer as defined in claim 18, wherein the mPEG-PLGA-L-phenylalanine is prepared by
reacting the mPEG-PLGA-RN-tert-butoxycarbonyl)-L-phenylalanine and trifluoroacetic acid in an organic solvent under nitrogen protection at about −20° C. to 40° C. for about 0.1 hour to 24 hours,
wherein a molar ratio of the mPEG-PLGA-[(N-tert-butoxycarbonyl)-L-phenylalanine to the trifluoroacetic acid is about 1:0.01 to 1:30.

22. The method for preparing the polyethyleneglycol-poly(lactic-co-glycolic acid)-poly-L-lysine polymer as defined in claim 18, wherein the PEG-PLGA-PLL cationic polymer is mPEG-PLGA-[(N-benzyloxycarboxylic)-PLL which is prepared by
reacting the mPEG-PLGA-L-phenylalanine and N-carboxyanhydrides of amino acids in an organic solvent under nitrogen protection at room temperature for about 1 to 6 days,
wherein a molar ratio of the mPEG-PLGA-L-phenylalanine to the N-carboxyanhydrides of amino acids is about 1:0.01 to 1:100.

23. The method for preparing the polyethyleneglycol-poly(lactic-co-glycolic acid)-poly-L-lysine polymer as defined in claim 18, wherein the mPEG-PLGA-PLL polymer is prepared by
reacting the mPEG-PLGA-[(N-benzyloxycarboxylic)-PLL and a 33% hydrobromic acid-glacial acetic acid solution at about 0° C. for about 0.1 to 24 hours,
wherein a molar ratio of the mPEG-PLGA-[(N-benzyloxycarboxylic)-PLL to the 33% hydrobromic acid-glacial acetic acid solution is about 1:0.01 to 1:100.

24. A drug delivery system, comprising the polyethyleneglycol-poly(lactic-co-glycolic acid)-poly-L-lysine polymer as defined in claim 15 in a form of particles as a skeleton,
wherein the polyethyleneglycol-poly(lactic-co-glycolic acid)-poly-L-lysine cationic polymer is modified with at least one or more targeting groups, carries at least one or more active substances, or both, and size of the particle is about 1 nm to 10 μm.

25. The drug delivery system as defined in claim 24, wherein the size of the particle is about 5 nm to 1000 nm.

26. The drug delivery system as defined in claim 24, wherein the targeting group is capable of being identified by a receptor on a tissue or a cell membrane and has suitable modifiable functional groups and derivatives thereof.

27. The drug delivery system as defined in claim 26, wherein the targeting group is a peptide inhibiting tumor angiogenesis, an anticancer angiogenesis factor, folic acid, an antibody, transferrin, a carbohydrate, a polysorbate, a polypeptide, glycyrrhizic acid, glycyrrhetinic acid, cholic acid, a low density lipoprotein, a hormone, or a nucleic acid.

28. The drug delivery system as defined in claim 27, wherein the anticancer angiogenesis factor is a fibroblast growth factors (FGFs) or a vascular endothelial growth factors (VEGF).

29. The drug delivery system defined in claim 24, wherein, the active substance is a drug, a gene, a contrast agent for diagnosis, or gas inside the microbubbles.

30. The drug delivery system defined in claim 29, wherein the drug is an anticancer drug capable of being made into a nano drug delivery system, a contrast agent for tumor diagnosis, or a diagnostic agent.

31. The drug delivery system as defined in claim 29, wherein, the gas inside the microbubbles is air, fluorocarbon gas, or sulfur hexafluoride.

32. A method of using the polyethyleneglycol-poly(lactic-co-glycolic acid)-poly-L-lysine polymer as defined in claim 15, wherein the polymer is applied as a drug or gene carrier.

33. The method of using as defined in claim 32, wherein the drug is a drug for injection, oral administration, or mucosal administration.

34. The method of using as defined in claim 32, wherein the drug is a targeting preparation of anticancer drugs, preparation to reverse or reduce the drug resistance of the tumor, a reagent for tumor diagnosis and contrast, a reagent to transfect DNA plasmids, a pharmaceutical preparation for cancer gene therapy, a reagent used to transfect antisense nucleic acid and siRNA, or a pharmaceutical preparation used to prepare antisense nucleic acid and siRNA.
